(19) United States
(12) Patent Application Publication
(10) Pub. No.: US 2026/0158071 A1
(43) Pub. Date: Jun. 11, 2026

(54) EXTRACELLULAR VESICLES FROM CHEMICAL HYPOXIA-PRECONDITIONED MESENCHYMAL STROMAL CELLS FOR THE TREATMENT OF VIRUS-INDUCED ACUTE LUNG INJURY

(71) Applicant: Centre for Immunology & Infection Limited, Hong Kong (HK)

(72) Inventors: Chi Wai Michael CHAN, Hong Kong (HK); Po Yee MA, Hong Kong (HK); Hiu Ha CHING, Hong Kong (HK)

(21) Appl. No.: 19/392,205

(22) Filed: Nov. 18, 2025

Related U.S. Application Data
(60) Provisional application No. 63/730,919, filed on Dec. 11, 2024.

(51) Int. Cl. A61K 35/28; A61K 31/215; A61K 31/351; A61K 31/7056; A61P 11/00; A61P 31/16; C12N 5/0775

(52) U.S. Cl. CPC A61K 35/28; A61K 31/215; A61K 31/351; A61K 31/7056; A61P 11/00; A61P 31/16; C12N 5/0665

(57) ABSTRACT

This invention provides a novel, cell-free treatment using extracellular vesicles (EVs) derived from chemical hypoxia-preconditioned umbilical cord mesenchymal stromal cells (UC-MSCs). In a physiologically relevant in vitro lung injury model, hypoxia MSC-EVs significantly restored AFC, reduced APP, suppressed proinflammatory cytokines, and enhanced ion transporter activity more effectively than normoxic EVs. In vivo, hypoxia MSC-EVs also improved survival and reduced viral burden in H5N1-infected mice. The invention demonstrates that hypoxic preconditioning markedly enhances the therapeutic potency of MSC-EVs against viral lung injury, offering an innovative and safer alternative to live cell therapies. This approach is particularly beneficial for treating late-phase acute lung injury in immunocompromised patients and represents an advancement over prior MSC or EV-based strategies.

### CROSS-REFERENCE TO RELATED APPLICATIONS

The present application claims the priorities from the U.S. provisional patent application Ser. No. 63/730,919 filed Dec. 11, 2024, and the disclosure of which is incorporated herein by reference in its entirety.

### FIELD OF THE INVENTION

The present invention relates to the field of regenerative medicine and respiratory disease therapeutics, and more particularly to a method for treating acute lung injury using extracellular vesicles derived from hypoxia-preconditioned mesenchymal stromal cells.

### BACKGROUND OF THE INVENTION

Acute lung injury (ALI), particularly when induced by severe viral infections such as highly pathogenic influenza viruses (e.g., H5N1), represents a critical clinical condition characterized by impaired alveolar fluid clearance (AFC), increased alveolar protein permeability (APP), excessive inflammation, and compromised gas exchange. These pathological changes often result in respiratory failure and are associated with high morbidity and mortality. Current treatments for viral ALI are largely supportive and include antiviral drugs, mechanical ventilation, and corticosteroids; however, their efficacy remains limited, especially during the late stages of disease progression when irreversible lung damage has occurred. Furthermore, existing therapies primarily target viral replication or symptomatic relief, but do not adequately promote tissue repair or modulate the host's inflammatory response.

Mesenchymal stromal cells (MSCs) have emerged as promising candidates in regenerative medicine due to their immunomodulatory effects, low immunogenicity, and ability to promote tissue repair. Recent studies have shifted focus from using MSCs themselves to leveraging their paracrine signaling components—collectively referred to as the MSC secretome—which includes soluble cytokines and extracellular vesicles (EVs). These EVs have been shown to mediate key therapeutic effects by transferring bioactive molecules to recipient cells. Despite these advantages, unmodified MSCs or EVs derived from normoxic cultures may exhibit inconsistent therapeutic outcomes due to variability in their composition and limited potency.

Preconditioning MSCs under hypoxic conditions, which simulate ischemic environments commonly observed in injured tissues, has been reported to enhance the therapeutic efficacy of the resulting secretome. To induce hypoxic conditions, besides physically reducing the oxygen in culture with the use of hypoxia chambers, hypoxia-mimetic agents that prevent hypoxia-inducible factor (HIF) degradation can provide higher oxygen tension stability. Specifically, hypoxia-induced EVs are enriched in angiogenic and regenerative factors that can potentially augment tissue repair and immunomodulation. However, there remains a lack of research focusing on the application of hypoxia-conditioned MSC-EVs in treating acute lung injury caused by respiratory viruses, particularly in physiologically relevant models that recapitulate human disease pathophysiology. In addition, the mechanisms underlying the enhanced therapeutic effects of such EVs remain insufficiently characterized.

Therefore, there is a need in the art for a safe, effective, and cell-free therapeutic strategy for treating acute lung injury caused by viral infections. Such a strategy should address the limitations associated with conventional MSC-based therapies, including risks of immune rejection, tumorigenicity, and manufacturing complexity. Ideally, the approach would mitigate inflammation, support epithelial recovery, and enhance antiviral responses, while avoiding the safety and regulatory challenges of live cell transplantation.

### SUMMARY OF THE INVENTION

Despite the recognized therapeutic benefits of MSCs, their clinical application remains hindered by several limitations, including the risk of immune rejection, tumorigenic potential of undifferentiated cells, and the possibility of transferring contaminated or infected cell cultures. Moreover, in vitro expansion of MSCs may lead to phenotypic drift and loss of functional potency. These safety and stability concerns restrict the direct use of MSCs in cell-based therapies for respiratory infectious diseases such as influenza-induced acute lung injury.

In view of these drawbacks, the present invention aims to provide a safer and more effective therapeutic strategy that harnesses the regenerative and immunomodulatory properties of MSCs without requiring live cell transplantation. Specifically, the invention utilizes EVs secreted by MSCs, which retain many of the parent cells'therapeutic functionalities while avoiding the complications associated with cellular therapies. The present invention addresses this need by providing a novel use of chemical hypoxia-preconditioned MSC-derived EVs and validating their efficacy through both in vitro and in vivo models of influenza-induced lung injury.

The invention discloses a method for enhancing the therapeutic efficacy of MSC-derived EVs by preconditioning umbilical cord-derived MSCs (UC-MSCs) under hypoxic conditions prior to EV isolation. Cobalt(II) chloride, the hypoxia-mimetic used here, prevents rapid degradation of hypoxia-inducible factor 1-alpha (HIF-1α) after removal of cobalt-containing medium. This allows for the experimental manipulation of hypoxic cells to be prolonged by several hours, overcoming the rapid HIF-1α degradation caused by reoxygenation that occurs when hypoxia chambers are opened. These hypoxia-preconditioned EVs (hypoxia MSC-EVs) are found to exhibit superior regenerative effects in both in vitro and in vivo models of respiratory virus-induced lung injury. A physiologically relevant human lung injury model was established using highly pathogenic influenza A(H5N1) virus to elucidate the protective mechanisms of the EVs on alveolar fluid clearance, alveolar protein permeability, inflammatory cytokine suppression, ion transporter restoration, and viral replication inhibition.

In a first aspect, the present invention provides an extracellular vesicle (EV) composition, which includes a population of EVs isolated from one or more MSCs cultured under chemically induced hypoxic conditions. The one or more MSCs are exposed to a hypoxia-mimetic agent prior to EV collection, the EVs exhibit a particle size distribution within a range of approximately 30 nm to approximately 150 nm in diameter and are characterized by a lipid bilayer membrane encapsulating a cytoplasmic protein cargo.

In accordance with one embodiment of the present invention, the cytoplasmic protein cargo includes at least CD9 and HIF-1α, and is substantially free of GM130.

In accordance with one embodiment of the present invention, the MSCs are umbilical cord-derived mesenchymal stromal cells.

In accordance with one embodiment of the present invention, the one or more MSCs are exposed to cobalt(II) chloride at a concentration of 50 to 200 μM for 48 to 96 hours. Alternative hypoxia-mimetics, such as deferoxamine (50 to 100 μM) and dimethyloxaloylglycine (0.5 to 1 mM), can also be used.

In accordance with one embodiment of the present invention, the EVs are obtained by sequential ultracentrifugation and 0.22 μm membrane filtration from conditioned medium.

In accordance with one embodiment of the present invention, the EVs include at least one protein selected from the group consisting of APOE, FGG, and NPTX1, wherein the expression level of the at least one protein is at least 2-fold higher than that in EVs derived from normoxia-cultured MSCs.

In accordance with one embodiment of the present invention, the EVs, when applied to H5N1-infected alveolar epithelial cells, reduce mRNA expression levels of at least one cytokine selected from TNF-α, IFN-β, and RANTES by at least 2-fold relative to untreated infected cells.

In accordance with one embodiment of the present invention, the EVs upregulate expression of ion transporters selected from the group consisting of epithelial sodium channels (ENaC), CFTR, and Na⁺/K⁺ ATPase.

In accordance with one embodiment of the present invention, the EVs induce ISG15 mRNA expression by at least 2-fold in virus-infected alveolar epithelial cells.

In accordance with one embodiment of the present invention, the EVs are further formulated in a pharmaceutically acceptable carrier suitable for intravenous administration.

In accordance with one embodiment of the present invention, the EVs are formulated in a dosage form selected from the group consisting of:
a lyophilized powder comprising one or more cryoprotectants selected from the group consisting of trehalose, sucrose, and mannitol;
a nebulizable aqueous solution formulated for pulmonary administration; and
one or more polymeric microparticles comprising the EVs encapsulated in a biodegradable polymer selected from the group consisting of poly(lactic-co-glycolic acid) (PLGA), chitosan, and hyaluronic acid.

In another aspect, the present invention provides a method for treating acute lung injury in a subject in need thereof, including administering to the subject an effective amount of the EV composition, wherein the lung injury is caused by viral infection, wherein the EVs are derived from MSCs cultured under chemically induced hypoxia using a hypoxia-mimetic agent.

In accordance with one embodiment of the present invention, the MSCs are exposed to cobalt(II) chloride at a concentration of 50 to 200 μM for 48 to 96 hours.

In accordance with one embodiment of the present invention, the EV composition is administered via an intravenous, intranasal, or intratracheal route.

In accordance with one embodiment of the present invention, the EVs reduce viral burden in a lung tissue by at least 50% relative to an untreated control.

In accordance with one embodiment of the present invention, the EVs reduce expression of TNF-α and RANTES by at least 2-fold compared to an untreated control.

In accordance with one embodiment of the present invention, the EVs restore alveolar fluid clearance (AFC) and reduce alveolar protein permeability (APP) within 24 hours post-administration.

In accordance with one embodiment of the present invention, the EVs increase ISG15 expression by at least 2-fold in infected alveolar epithelial cells.

In accordance with one embodiment of the present invention, the EV composition is co-administered with one or more antiviral agents comprising oseltamivir, zanamivir, or ribavirin.

In accordance with one embodiment of the present invention, the effective amount of the EV composition comprises a dose of approximately 1×10⁸ to 1×10¹² EV particles per administration.

In accordance with one embodiment of the present invention, the EV composition is administered in multiple doses over a period of 3 to 10 days following onset of viral infection symptoms.

Unexpectedly, the hypoxia MSC-EVs demonstrated significantly improved therapeutic performance compared to normoxia-derived EVs, including greater reversal of H5N1-induced AFC impairment, enhanced restoration of ion transporter activity, stronger upregulation of antiviral gene expression, and more effective suppression of inflammatory cytokines and viral load. These pronounced effects were further validated in a murine model of H5N1 infection, where hypoxia MSC-EV treatment significantly improved survival outcomes and reduced pathological weight loss.

In contrast to prior approaches utilizing unmodified EVs or MSCs directly, the present invention demonstrates that preconditioning UC-MSCs under chemically induced hypoxic conditions (e.g., using cobalt(II) chloride) substantially alters the EV protein cargo, leading to significant therapeutic advantages. These advantages include enhanced suppression of proinflammatory cytokines (e.g., TNF-α, IFN-β), improved ion transporter recovery (e.g., ENaC, CFTR), and stronger upregulation of antiviral genes (e.g., ISG15), none of which are anticipated by the prior art.

### BRIEF DESCRIPTION OF THE DRAWINGS

Embodiments of the invention are described in more details hereinafter with reference to the drawings, in which:

FIGS. 1A-1D shows isolation of EVs derived from mesenchymal stromal cells (MSC-EVs) from cell culture medium. (FIG. 1A) Schematics of using differential centrifugation to isolate MSC-EVs from hypoxia-treated MSC culture medium, which contained secreted EVs and proteins. Characterization of EVs was performed by (FIG. 1B) western blotting, (FIG. 1C) nanoparticle tracking analysis and (FIG. 1D) transmission electron microscopy. N=normoxia, H=hypoxia, GM130=negative EV marker, CD9=positive EV marker, β-actin=loading control and HIF-1α=hypoxia marker;

FIGS. 2A-2G show hypoxia-conditioned MSC-EVs have a more pronounced effect in preventing influenza A(H5N1) virus impairment of alveolar epithelial AFC and APP than normal MSC-EVs in vitro. (FIG. 2A) Schematics of the in vitro lung injury model used for studying AFC and APP. AECs seeded in a transwell were first infected with H5N1 influenza virus, then treated with either normoxia or hypoxia MSC-EVs. EV treatment of infected cells restored (FIG. 2B) AFC and decreased (FIG. 2C) APP compared to untreated infected cells. H5N1-infected cells displayed overexpression of (FIG. 2D) pro-inflammatory cytokines and suppressed (FIG. 2E) ion transporter activity; EV treatment reversed these effects. (FIG. 2F) Further upregulation of anti-viral genes with EV treatment post-infection may be responsible for (FIG. 2G) the suppression of infectious viral titer by EVs. Data represent the mean ±SD from three experiments. *P≤0.05, **P≤0.01 and ***P≤0.001;

FIGS. 3A-3G show hypoxia MSC-EVs significantly reduced weight loss of H5N1-infected mice in vivo. (FIG. 3A) Schematics of intranasal H5N1 infection of mice and subsequent intravenous administration of MSC-EVs for treatment. (FIG. 3B) Hypoxia MSC-EV significantly prevented weight loss induced by H5N1. (FIG. 3C) Survival of H5N1-infected mice significantly increased with MSC-EV treatment. Pro-inflammatory cytokine levels in (FIG. 3D) lung homogenate and (FIG. 3E) bronchoalveolar lavage (BAL) fluid were suppressed by MSC-EVs. Infectious viral titers significantly decreased in MSC-EV treated mice as shown by (FIG. 3F) influenza viral M-gene expression and (FIG. 3G) TCID assay. Data represent the mean ±SD from three experiments. *P≤0.05; **P≤0.01; ***P≤0.001 and ****P≤0.0001; and

FIGS. 4A-4C shows proteomics profiling revealed the nature of hypoxia-conditioned MSC-EVs compared to normoxia-cultured MSC-EVs. (FIG. 4A) Upregulated and downregulated proteins in hypoxic MSC-EVs compared to normoxic MSC-EVs. (FIG. 4B) Cellular components related to hypoxic MSC-EV proteins. (FIG. 4C) Molecular functions related to hypoxic MSC-EV proteins.

### DETAILED DESCRIPTION

### Definitions

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they allow for elements not explicitly recited, but exclude elements that are found in the prior art or that affect a basic or novel characteristic of the present invention.

Furthermore, throughout the specification and claims, unless the context requires otherwise, the word “include” or variations such as “includes” or “including”, will be understood to imply the inclusion of a stated integer or group of integers but not the exclusion of any other integer or group of integers.

As used herein and not otherwise defined, the terms “substantially,” “substantial,” “approximately” and “about” are used to describe and account for small variations. When used in conjunction with an event or circumstance, the terms can encompass instances in which the event or circumstance occurs precisely as well as instances in which the event or circumstance occurs to a close approximation. For example, when used in conjunction with a numerical value, the terms can encompass a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

References in the specification to “one embodiment”, “an embodiment”, “an example embodiment”, etc., indicate that the embodiment described can include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

As used herein, the term “extracellular vesicle” (EV) refers to a lipid bilayer-enclosed, nano-sized vesicle naturally secreted by cells into the extracellular space. The EVs of the present invention typically have a diameter ranging from approximately 30 to 150 nanometers, exhibit spherical morphology, and are substantially free of cellular organelle contaminants. The EVs may contain proteins, RNAs, lipids, or other bioactive molecules derived from the originating cell and are capable of modulating biological activity in recipient cells.

The term “hypoxia-preconditioned MSC” or “hypoxia MSC” refers to a mesenchymal stromal cell that has been cultured under chemically induced hypoxic conditions, such as by exposure to cobalt(II) chloride at concentrations between 50 to 200 μM for a duration of 48 to 96 hours, thereby simulating low-oxygen conditions and enhancing the secretion of extracellular vesicles enriched in therapeutic factors.

The term “pharmaceutically acceptable carrier” refers to any biocompatible vehicle or excipient that can be used to formulate the extracellular vesicle composition for therapeutic administration without causing significant adverse effects. Exemplary carriers include, but are not limited to, saline, phosphate-buffered saline (PBS), aqueous buffers, cryoprotectant solutions, or biodegradable polymers.

The term “acute lung injury” (ALI) refers to a pathological condition of the lungs characterized by impaired alveolar fluid clearance (AFC), increased alveolar protein permeability (APP), epithelial barrier dysfunction, and excessive inflammatory response, often resulting from infection with respiratory viruses such as influenza A(H5N1).

The term “effective amount” refers to an amount of the extracellular vesicle composition sufficient to produce a measurable biological response or therapeutic benefit in the subject being treated, such as reduction of inflammation, improvement of AFC, suppression of viral burden, or increase in survival rate.

Other definitions for selected terms used herein may be found within the detailed description of the present invention and apply throughout. Unless otherwise defined, all other technical terms used herein have the same meaning as commonly understood to one of ordinary skill in the art to which the present invention belongs.

The present invention provides a novel, cell-free therapeutic approach for treating acute lung injury caused by respiratory pathogens. By leveraging hypoxia-induced enhancement of MSC-EV functionality, the invention overcomes key limitations of conventional MSC-based therapies.

Respiratory virus infections caused by highly pathogenic strains, such as influenza A(H5N1), are associated with high mortality and limited treatment options. Existing antiviral therapies often fail to prevent disease progression, especially when administered at later stages. Survivors frequently suffer from long-term pulmonary sequelae including reduced lung function and persistent fatigue due to epithelial barrier damage. Therefore, a therapeutic approach that facilitates functional recovery of alveolar structures is urgently needed.

Extracellular vesicles (EVs) are lipid bilayer-enclosed nanovesicles naturally secreted by living cells. These vesicles function in intercellular communication by transferring bioactive cargoes such as proteins, RNAs, and lipids to recipient cells. The uptake of EV contents alters intracellular signaling and modulates recipient cell phenotype. In particular, MSC-EVs have demonstrated immunomodulatory, anti-inflammatory, and tissue-reparative effects in various preclinical models. Their application as a cell-free therapeutic is especially attractive due to their low immunogenicity and stability.

Accordingly, the present invention relates to a therapeutic composition comprising EVs derived from MSCs that have been preconditioned under hypoxic conditions. The composition is suitable for treating acute lung injury (ALI) resulting from respiratory viral infections. In particular, the invention provides a cell-free therapeutic modality capable of modulating proinflammatory responses, restoring epithelial ion transport, and enhancing antiviral gene expression in damaged lung tissue.

In one embodiment, the MSCs are subjected to hypoxia preconditioning by chemical induction using cobalt(II) chloride at a concentration of 50 to 200 μM for 48 to 96 hours. This hypoxic exposure enhances the secretory activity of the MSCs and alters the composition of the released EVs. Compared to normoxia-derived EVs, the EVs obtained from hypoxia-preconditioned MSCs (hypoxia MSC-EVs) exhibit a distinct proteomic profile and enhanced biological activity. The resulting EVs are collected from the conditioned medium using a sequential ultracentrifugation protocol followed by filtration through a 0.22 μm membrane.

In one embodiment, the hypoxia MSC-EVs are characterized by a spherical morphology, a particle diameter of 30 to 150 nanometers, and the presence of surface marker CD9. The vesicles encapsulate HIF-1α within their protein cargo, and are substantially devoid of intracellular organelle markers such as GM130, indicating high purity. These EVs are capable of modulating inflammatory signaling and epithelial fluid transport functions.

In one embodiment, the therapeutic effect of the EV composition is evaluated using a physiologically relevant in vitro model of virus-induced acute lung injury. Primary human alveolar epithelial cells (AECs) are cultured on transwell membranes and infected with a highly pathogenic strain of influenza A(H5N1). Viral infection results in impaired alveolar fluid clearance (AFC), increased alveolar protein permeability (APP), and elevated expression of proinflammatory cytokines including TNF-α, IFN-β, and RANTES. These pathological changes are accompanied by reduced expression of key ion transporters such as ENaC, CFTR, and Na⁺/K⁺ ATPase.

In one embodiment, treatment with hypoxia MSC-EVs results in significant restoration of AFC and suppression of APP. The EV-treated AECs demonstrate increased expression of epithelial ion transporters and decreased levels of proinflammatory cytokines. Additionally, treatment induces upregulation of antiviral gene ISG15 and leads to a reduction in viral burden. These effects are more pronounced with hypoxia MSC-EVs than with normoxia-derived EVs, indicating enhanced therapeutic efficacy.

In another embodiment, the therapeutic efficacy of hypoxia MSC-EVs is confirmed in vivo using a murine model of H5N1-induced lung injury. Mice are infected intranasally with H5N1 virus and treated intravenously with either normoxia or hypoxia MSC-EVs at defined timepoints following symptom onset. Mice treated with hypoxia MSC-EVs exhibit improved clinical outcomes, including reduced body weight loss, increased survival rates, and decreased viral titers in lung tissue. Analysis of bronchoalveolar lavage fluid (BALF) and lung homogenates reveals a more substantial suppression of inflammatory cytokines in the hypoxia EV-treated group, particularly TNF-α.

Taken together, these findings demonstrate that extracellular vesicles derived from hypoxia-preconditioned MSCs constitute a structurally and functionally optimized composition for the treatment of virus-induced acute lung injury. The EVs exhibit enhanced bioactivity in modulating inflammatory, epithelial, and antiviral responses, thereby providing a clinically translatable, cell-free therapeutic alternative for managing both early-stage and late-phase respiratory viral pathology.

### EXAMPLE

### Example 1—Materials and Methods

### Virus

Influenza virus A/HK/483/97 (H5N1) was used for in vitro infection. All influenza viruses were passaged in Madin-Darby Canine Kidney (MDCK) cells. Viral titers were determined by median tissue culture infectious dose (TCID₅₀). All experiments were performed inside a biosafety level-3 facility.

### Culture of UC-MSCs

Umbilical Cord-MSCs were isolated by HealthBaby Biotech (Hong Kong) Company Ltd. and cultured in low glucose (1.0 g/L) Dulbecco's modified Eagle's medium (DMEM) (Gibco, USA) supplemented with 10% HyClone fetal bovine serum (FBS) (SV30160.03, Thermo Fisher) and 1% P/S was used for cell culture. UC-MSCs were cultured in humidified atmosphere (37° C., 5% CO₂) with growth medium changed every 48-72 hours. Cells were trypsinized and seeded for experimental use upon 70% confluency.

### Isolation of EVs From UC-MSCs

At 70% confluency, UC-MSCs were washed 3 times with HBSS to remove HyClone FBS, then replenished with 10% EV-depleted FBS low glucose DMEM. To generate hypoxia cell culture, 100 μM cobalt(II) chloride was added to the culture medium. Supernatant was collected at 72 h and stored at −20° C. until EV isolation. Differential centrifugation was employed to isolate EVs with the following steps: 1) centrifugation at 3,000 rpm for 15 minutes at 4° C.; 2) harvest supernatant and centrifuge again at 20,000g for 30 minutes at 4° C.; 3) harvest supernatant, pass it through a 0.22 μM filter, and centrifuge at 100,000 g for 2 hours at 4° C.; 4) discard supernatant, wash EV pellet with PBS and centrifuge EV suspension for another 2 hours at 100,000g at 4° C.; 5) remove supernatant, resuspend EV pellet in PBS and store at −20° C. until use.

### Isolation and Culture of Primary Human AECs

AECs were isolated from resected, non-malignant lung tissue obtained from the Department of the Cardiothoracic Surgery, Queen Mary Hospital, Hong Kong. Firstly, visible bronchi were removed and lung tissue was subject to mincing into pieces of >0.5 mm thickness using scissors. Minced lung pieces were washed with Hank's balanced salt solution at pH 7.4 (Invitrogen, USA) to partially remove macrophages and blood cells. Washed lung pieces were then digested using 0.5% trypsin (GibcoBRL, USA) and 4 U/ml elastase (Worthington Biochemical, USA) for 40 minutes (mins) at 37° C. in a shaking water-bath. Digestion was terminated with 40% FBS DMEM/F 12 medium and DNase I (350 units/ml) (Sigma, USA). Incompletely digested lung was separated using a cell strainer of 50 μM pore size (BD Bioscience, USA). Cell clumps were dispersed by repeatedly pipetting for 10 mins. After spinning down, cells were incubated with a 1:1 mixture of DMEM/F 12 medium and small airway growth medium (SAGM) (Lonza, USA) containing 5% FBS and 350 units/ml DNase I. Cells were seeded on a new tissue culture flask (Corning, USA) for adhesion at 37° C. Cells that did not adhere were collected for centrifugation and resuspended with SAGM in a new tissue culture flask. Culture medium was changed daily within the first 4 days of plating. AECs were trypsinized for seeding upon 75% confluency.

### In Vitro Acute Lung Injury Model

To study the effect of influenza viruses on alveolar fluid clearance and protein permeability in human alveolar epithelial cells, a physiologically relevant 24-transwell in vitro acute lung injury model was used. The alveolar epithelial cells were plated on the apical chamber of 24-well Costar Transwell inserts with a 0.4-μm pore size (Corning), at a density of 1×10⁵ cells per well. The microporous transwell membrane established a liquid-liquid interface similar to that of human lung epithelium, and the plated cells were maintained in a humidified atmosphere (5% CO₂, 37° C.). Transepithelial resistance was maintained at ≥800 Ω/cm², which indicates good tight junction integrity between cells.

### Measurement of Alveolar Fluid Clearance and Alveolar Protein Permeability

Net alveolar fluid transport from the apical chamber of the transwell culture insert (containing a monolayer of alveolar epithelial cells infected with influenza virus) to the basolateral chamber of the transwell at 24 hours post infection was measured. Alveolar epithelial cells were inoculated with the respective influenza A viruses at a multiplicity of infection (MOI) of 0.1 for 1 hour. Then, 200 μl of FITC-labeled dextran (Sigma) with a size of 70 kDa was added to the cells (final concentration, 500 ng/μl). After 5 minutes, a sample of 100 μl was collected from the apical chamber for initial FITC measurement, after which it is transferred back into the apical chamber for overnight incubation. After 24 hours of dextran incubation, 100 μl was collected from the apical chamber, while 100 μl was collected from the basolateral chamber for the final FITC measurement. The fluorescence of each sample was measured by a modulus fluorometer (FLUOstar OPTIMA, BMG Labtech) at excitation wavelength of 485 nm and emission wavelength of 520 nm. A standard curve of known FITC concentration was constructed for the calculation of FITC-dextran present in the transwell chambers at different time points. The net alveolar fluid transport across the epithelial monolayer was measured as [1 - (FITC concentration in the initial apical sample/FITC concentration in the final apical sample)]/200 μl/0.33 cm²/24 hours. The final basolateral reading was collected after 24 hours of dextran incubation to measure protein permeability, based on the unidirectional flux of fluorescence-labeled dextran from the apical to the basolateral chamber of the transwell culture insert.

### Virus Infection of Mice

6-8 weeks old female BALB/c mice were intranasally inoculated with 10⁶ log TCID₅₀ of A/Hong Kong/486/1997 (H5N1) influenza virus in 25 μl volume. On days 3, 5 and 7 post infection, mice were injected intravenously with 1×10⁸ MSC-EVs or PBS in 100 μl. Survival and body weight were monitored for 10 days. On day 10 post infection, virus was titrated in three mouse lungs per group. BAL fluid was collected for cytokine assay using a mouse Luminex assay according to the manufacturer's instruction manual (R&D Systems, USA). Also, three mouse lungs per group were fixed for histopathological analysis.

Quantification of Influenza Matrix Gene, Inflammatory Cytokines, Anti-Viral Genes and Ion Transporter mRNA by Quantitative RT-PCR

Total RNA and cellular protein were extracted from infected AEC at 24 hours post infection. RNA extraction was performed, and the RNA was then reverse transcribed into complementary DNA using PrimeScript RT Reagent Kit (TaKaRa, Dalian), both according to manufacturer's instructions. AECs and MSCs infected with influenza viruses at MOI 2 were lysed with 350 μl buffer RLT (Qiagen, Germany) with beta-mercaptoethanol (Sigma, USA). Extraction of RNA was carried out using the MiniBEST Universal RNA extraction kit (TaKaRa, Dalian) with DNase treatment as per manufacturer's instructions. PrimeScript RT reagent kit (TaKaRa, China) was used for reverse transcription. ViiA™ 7 Real-Time PCR System (Applied Biosystem, USA) was used to perform real-time PCR. Gene expression profiles were normalized with housekeeping gene β-actin mRNA. Standard plasmid with a known copy number was run in conjunction with gene of study for the generation of a standard curve to determine absolute copy numbers. Master mix including Power SYBR Green PCR Master Mix (Applied Biosystem, USA) with cDNA reverse-transcribed from 500 ng of total RNA, were amplified by real-time PCR for 40 cycles with an ABI 7500 PCR system (Applied Biosystem, USA). Gene expression and statistical analysis were calculated following the instructions provided by the manufacturer.

### Statistical Analysis

All in vitro experiments were conducted independently in triplicates. Groups were compared by using an unpaired, two-tailed t test. Differences were considered significant if P≤0.05.

### Example 2—Isolation of MSC-EVs From MSC Secretome

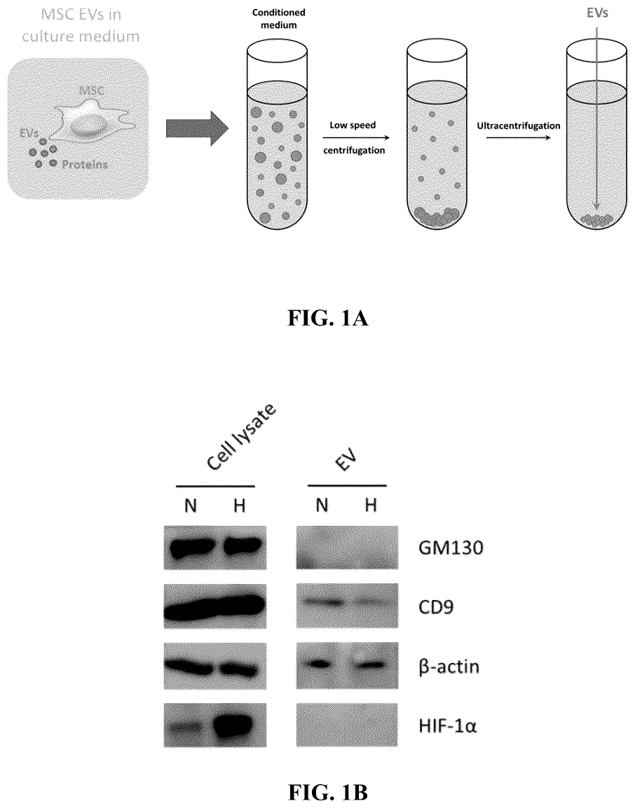

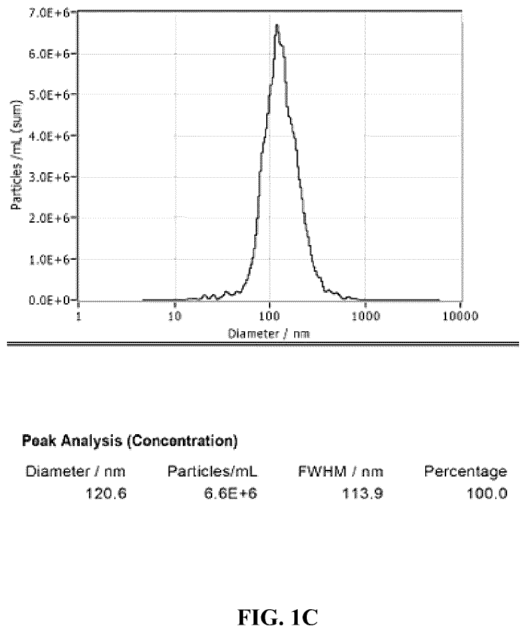

Umbilical cord-derived mesenchymal stromal cells (UC-MSCs) were cultured under either normoxic (21% O₂) or chemically induced hypoxic conditions to investigate the effect of oxygen availability on extracellular vesicle (EV) production. Hypoxia was induced by supplementing the culture medium with 100 μM cobalt(II) chloride (CoCl₂), a well-established hypoxia mimetic agent. Culture medium was prepared using low-glucose Dulbecco's Modified Eagle Medium (DMEM) supplemented with 10% EV-depleted fetal bovine serum (FBS). Cells were incubated for 72 hours under the respective conditions prior to EV harvesting. A schematic representation of the EV isolation procedure is shown in FIG. 1A.

Following hypoxic or normoxic incubation, the conditioned medium containing secreted EVs and soluble proteins was harvested and subjected to a multi-step differential centrifugation protocol for the selective isolation of EVs, as schematically illustrated in FIG. 1A. This purification strategy was designed to maximize yield and purity of small EVs while minimizing contamination from cellular debris and soluble protein aggregates. The isolation procedure included:
(i) a low-speed centrifugation at 3,000 rpm for 15 minutes at 4° C. to remove cell debris and apoptotic bodies;
(ii) a medium-speed centrifugation at 20,000×g for 30 minutes to eliminate larger vesicles and macromolecular complexes;
(iii) filtration of the supernatant through a 0.22 μm pore-size membrane to remove particles exceeding the EV size range; and
(iv) ultracentrifugation at 100,000×g for 2 hours to pellet the EVs.

To ensure removal of residual soluble proteins and to further concentrate EVs, the pellet was washed once with phosphate-buffered saline (PBS) and subjected to a second round of ultracentrifugation under identical conditions. The final EV pellet was resuspended in PBS and stored at −20°C until use in downstream characterization and functional studies. This protocol combines sequential density-and size-based fractionation with two-step ultracentrifugation, allowing for the reproducible isolation of morphologically intact EVs with minimal contamination. Compared to conventional single-step ultracentrifugation methods, the approach described herein provides enhanced EV purity and experimental consistency, which are essential for downstream molecular profiling and therapeutic applications.

Characterization of the isolated EVs confirmed the successful recovery of high-purity, functionally active small EVs. As shown in FIG. 1B, Western blot analysis revealed strong expression of the canonical EV marker CD9 in both normoxia-and hypoxia-derived EVs, while the endoplasmic reticulum protein GM130, a known negative marker for EV contamination, was absent in all EV preparations. The absence of GM130 confirms the exclusion of intracellular organelles and validates the purity of the EV fraction. In parallel, expression of HIF-1α was observed in cell lysates and corresponding hypoxia-derived EVs, confirming that hypoxic preconditioning of the MSCs was effectively induced and that functional hypoxia-related proteins may be carried over into the secreted EVs. β-actin was used as an internal loading control.

Nanoparticle tracking analysis (NTA) was employed to determine particle size distribution and concentration of the isolated EVs (FIG. 1C). The particle population exhibited a unimodal size distribution centered at approximately 120.6 nm, with a full-width at half maximum (FWHM) of 113.9 nm. The particle concentration was determined to be 6.6×10⁶ particles/mL, indicating a robust EV yield. Notably, the size range is consistent with the definition of small EVs (typically <150 nm), and the sharp peak suggests a highly homogeneous vesicle population, which enhances batch-to-batch reproducibility for therapeutic applications.

Transmission electron microscopy (TEM) further confirmed the vesicular morphology and ultrastructural integrity of the isolated particles (FIG. 1D). The EVs appeared as spherical, lipid bilayer-enclosed structures with diameters below 150 nm, consistent with exosome-like morphology. The high-resolution imaging at 100 kV and ×28,500 magnification also revealed the absence of vesicle aggregation or membrane disruption, further supporting the structural quality and physical stability of the EV preparations.

Taken together, the combined biochemical (Western blot), biophysical (NTA), and TEM analyses validate that the EVs isolated using the disclosed protocol exhibit key physicochemical features consistent with functional small EVs. Moreover, the presence of hypoxia-inducible proteins within hypoxia-derived EVs provides molecular evidence of their enhanced bioactivity, supporting their use as a novel class of therapeutically active nanovesicles with improved potency over unconditioned EVs.

### Example 3—In vitro Restoration of AFC and APP by Hypoxia MSC-EVs

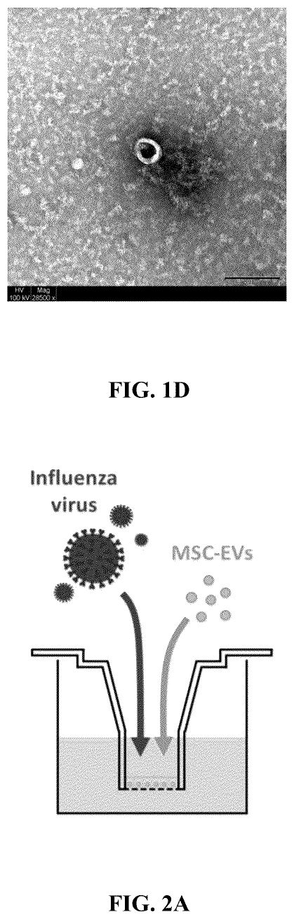

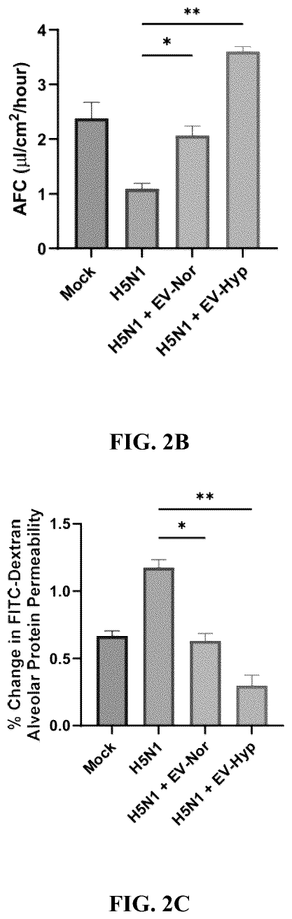

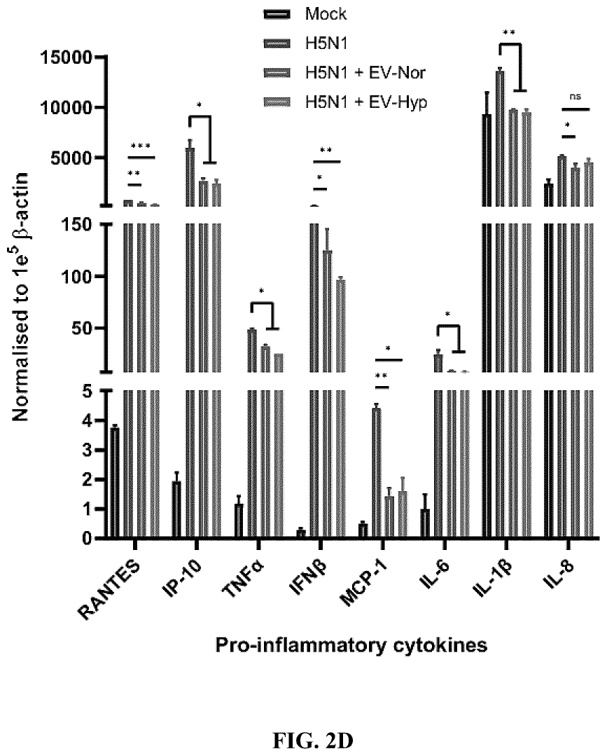

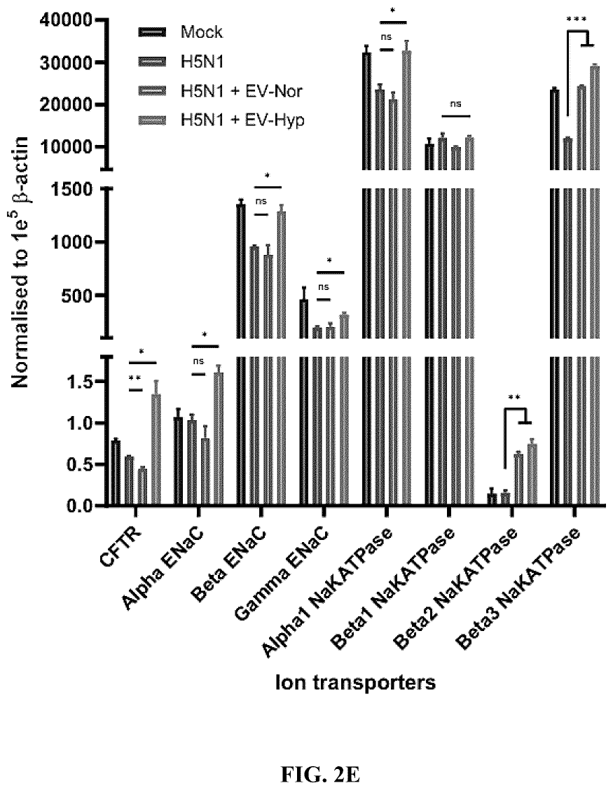

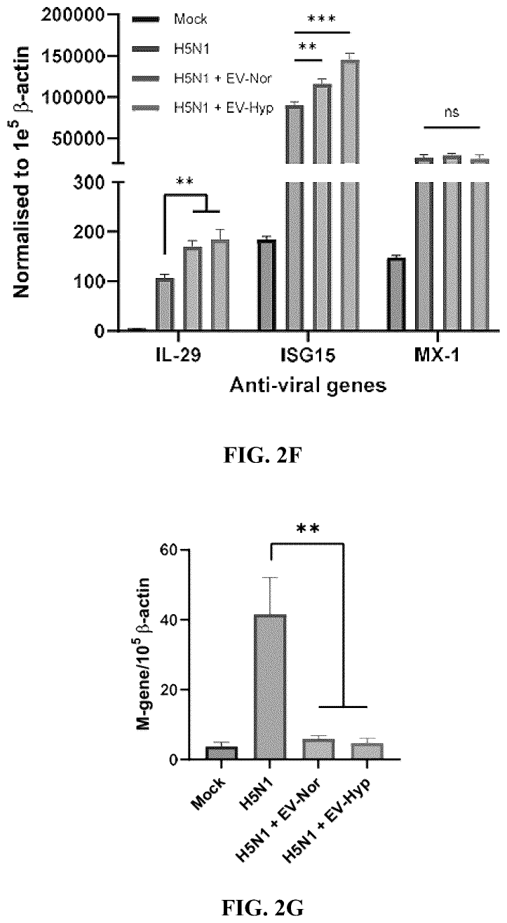

To evaluate the therapeutic effects of extracellular vesicles (EVs) derived from hypoxia-preconditioned mesenchymal stromal cells (MSC-EVs), a physiologically relevant in vitro model of influenza virus-induced alveolar injury was established, as illustrated in FIG. 2A. In this model, primary human alveolar epithelial cells (AECs) were cultured on the apical side of porous transwell culture inserts, forming a monolayer that mimics the human alveolar barrier. The cells were grown to confluence under air-liquid interface conditions and were validated to possess intact tight junctions, as confirmed by transepithelial electrical resistance (TEER) values exceeding 800 Ω·cm². This threshold indicates the formation of a functionally competent epithelial barrier suitable for modeling vectorial fluid transport and epithelial leakage.

The experimental setup allowed for the controlled application of both viral insult and therapeutic intervention on the apical side of the AEC monolayer, thereby simulating the physiological direction of airborne respiratory pathogen exposure and therapeutic delivery. Specifically, H5N1 viruses and MSC-EVs were administered directly to the apical chamber, enabling simultaneous infection and treatment in a spatially confined and reproducible environment. This model offers several advantages over conventional submerged cultures, including directional cytokine sampling, quantification of alveolar fluid clearance (AFC), and alveolar protein permeability (APP), all of which are clinically relevant endpoints in acute lung injury. Compared to prior in vitro approaches that rely on undifferentiated or immortalized cell lines, the use of primary human AECs in a transwell insert system provides improved biological fidelity, making it a robust platform for evaluating the barrier-restorative, immunomodulatory, and antiviral properties of therapeutic EVs in the context of viral lung epithelial injury.

To evaluate the effects of MSC-derived EVs on epithelial barrier function following viral injury, primary human alveolar epithelial cells (AECs) were infected with highly pathogenic influenza A/H5N1 virus at a multiplicity of infection (MOI) of 0.1. At 24 hours post-infection, key physiological parameters were measured, including AFC and APP, both of which reflect the integrity and functionality of the alveolar barrier.

As shown in FIG. 2B, H5N1 infection led to a marked reduction in AFC, indicating compromised ion transport and fluid reabsorption across the epithelial monolayer. Conversely, treatment with MSC-EVs restored AFC toward baseline levels. Notably, EVs derived from hypoxia-preconditioned MSCs (EV-Hyp) resulted in significantly greater recovery of AFC compared to normoxia-derived EVs (EV-Nor), achieving AFC values that exceeded even those of the uninfected mock group. This unexpected enhancement suggests that hypoxia EVs not only mitigate viral damage but also promote active fluid transport more effectively than standard EV formulations. Similarly, H5N1 infection induced a pronounced increase in alveolar protein permeability, as measured by the percentage change in FITC-dextran translocation (FIG. 2C). This reflects a breakdown in epithelial tight junctions and mimics the pathophysiological features of acute lung injury. Treatment with MSC-EVs significantly reduced protein leakage, with hypoxia-derived EVs achieving the most substantial suppression of APP among all groups tested. The reduction was statistically significant compared to both the untreated and EV-Nor groups, underscoring the functional superiority of hypoxia EVs in restoring epithelial barrier integrity. These findings demonstrate that the use of hypoxia-preconditioned MSC-EVs confers measurable and unexpectedly enhanced therapeutic effects on two clinically relevant epithelial functions—fluid absorption and barrier tightness—that are commonly impaired during severe respiratory viral infections. The dual restoration of AFC and APP underlines the multifaceted mechanism of action of hypoxia MSC-EVs and distinguishes them from conventional EV treatments in terms of potency and physiological relevance.

H5N1 infection also led to the upregulation of pro-inflammatory cytokines, including TNF-α, IFN-β, and RANTES, and downregulation of ion transporters such as ENaC, CFTR, and Na⁺/K⁺-ATPase. MSC-EV treatment reversed these gene expression changes, with hypoxia MSC-EVs inducing a more pronounced suppression of cytokines and restoration of transporter expression (FIGS. 2D-2E). Furthermore, MSC-EVs upregulated antiviral gene expression, including ISG15, with hypoxia EVs again demonstrating stronger induction than normoxia EVs (FIG. 2F). Both EV groups suppressed viral titers in infected AECs, but hypoxia MSC-EVs yielded significantly greater antiviral activity (FIG. 2G).

These findings establish that hypoxia MSC-EVs possess enhanced therapeutic activity in vitro, capable of restoring epithelial function, mitigating inflammation, and activating antiviral responses in influenza-infected human alveolar epithelial cells. The magnitude and breadth of these effects were unexpected and not suggested by the prior art involving conventional MSC or EV-based interventions.

### Example 4—In Vivo Therapeutic Evaluation of Hypoxia MSC-EVs in H5N1-Infected Mice

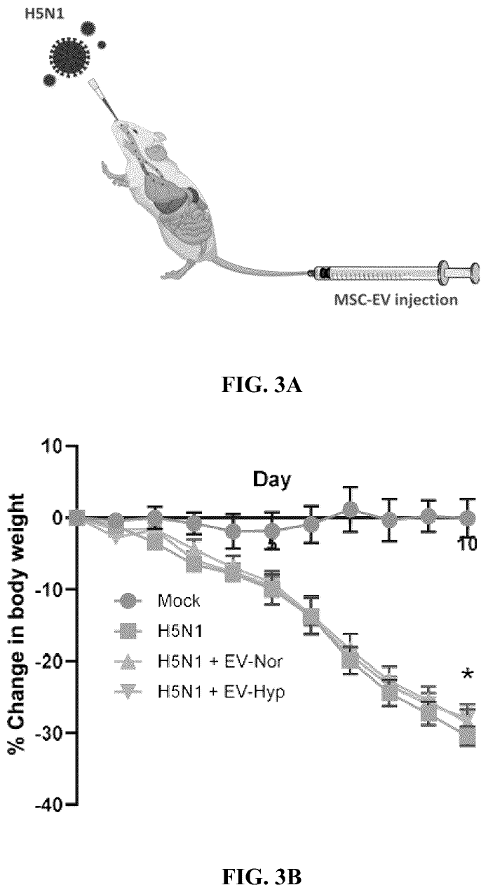

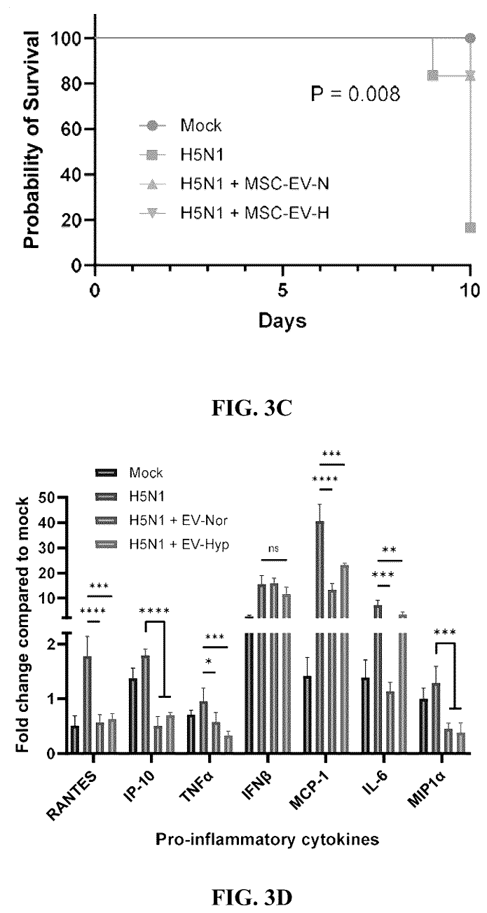

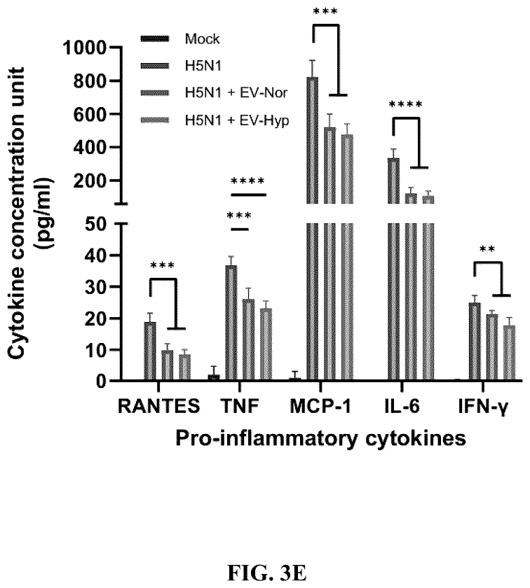

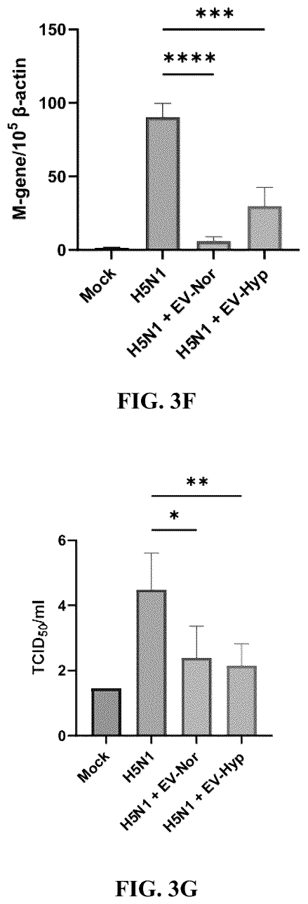

To further substantiate the therapeutic potential of hypoxia-preconditioned MSC-derived extracellular vesicles (hypoxia MSC-EVs) in a physiologically relevant disease context, an in vivo model of virus-induced acute lung injury was established in mice, as illustrated in FIG. 3A. Female BALB/c mice (6-8 weeks old) were intranasally inoculated with 10⁶ TCID₅₀ of the highly pathogenic human-derived influenza A virus strain A/Hong Kong/486/1997 (H5N1) in a 25 μL volume under light anesthesia. This route of infection accurately mimics the natural transmission of airborne respiratory viruses and selectively targets the lower respiratory tract, including the alveolar regions.

Following infection, the animals were monitored for the development of clinical signs such as weight loss, respiratory distress, and reduced activity. Upon onset of overt symptoms—representing a clinically relevant therapeutic window rather than a prophylactic setting—mice received intravenous injections of MSC-EVs on days 3, 5, and 7 post-infection. The treatment groups included EVs derived from either normoxic (EV-Nor) or hypoxia-preconditioned (EV-Hyp) MSC cultures, standardized to a dosage of 1×10⁸ particles per injection. This post-symptomatic dosing strategy distinguishes the present study from prior prophylactic EV administrations, reflecting a more stringent and translationally meaningful evaluation of therapeutic efficacy. Moreover, the systemic (intravenous) delivery route enables the EVs to circulate and reach the pulmonary vasculature, facilitating targeted uptake by inflamed or injured alveolar tissues. The combination of a high-virulence viral strain, clinically synchronized treatment onset, and comparative EV subtypes in a rigorously controlled murine model enables the assessment of not only survival benefit but also the mechanistic impact of hypoxia MSC-EVs on inflammation resolution, viral clearance, and lung tissue recovery.

Body weight and survival were monitored for 10 days following infection. Mice treated with hypoxia MSC-EVs exhibited significantly reduced weight loss compared to untreated controls (FIG. 3B) and showed a higher survival rate over the course of observation (FIG. 3C). Inflammatory cytokine levels were measured in lung homogenates and bronchoalveolar lavage (BAL) fluid, revealing that MSC-EV treatment reduced H5N1-induced pro-inflammatory responses, with hypoxia MSC-EVs exerting a more substantial suppressive effect, particularly on TNF-α expression (FIGS. 3D-3E).

Viral burden was evaluated through M-gene expression analysis and tissue culture infectious dose (TCID) assays. Both normoxia and hypoxia MSC-EVs effectively suppressed viral replication in lung tissue, with hypoxia MSC-EVs displaying superior antiviral efficacy (FIGS. 3F-3G).

These in vivo results confirm that hypoxia-preconditioned MSC-EVs not only mitigate clinical symptoms and reduce mortality in H5N1-infected mice but also attenuate pulmonary inflammation and lower viral burden more effectively than normoxic MSC-EVs. These outcomes provide compelling evidence for the enhanced therapeutic utility of hypoxia MSC-EVs in treating respiratory virus-induced acute lung injury in a living organism.

### Example 5—Proteomic Characterization of Hypoxia-Preconditioned MSC-EVs

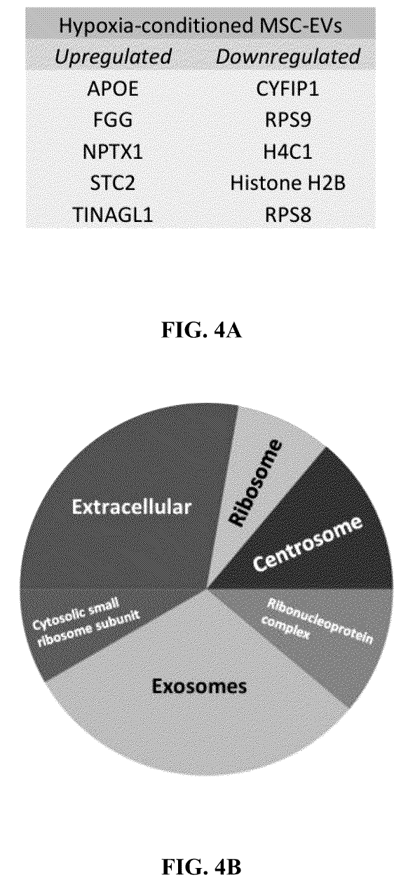

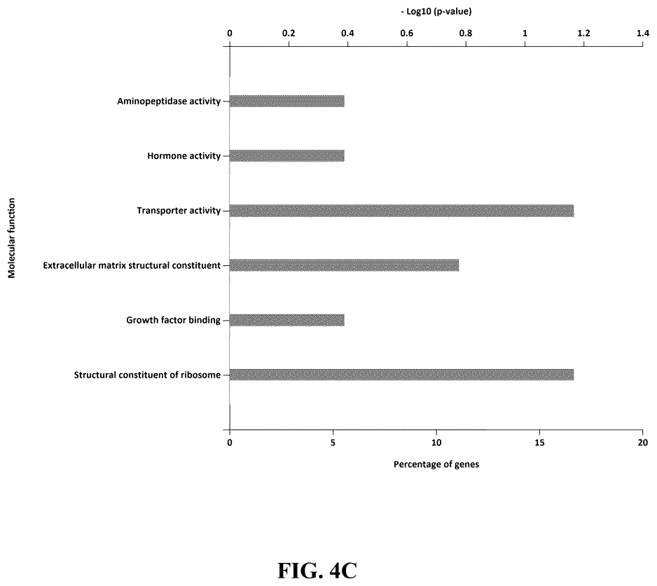

To elucidate the molecular basis underlying the enhanced therapeutic performance of hypoxia-preconditioned MSC-derived extracellular vesicles (hypoxia MSC-EVs), comparative proteomic profiling was conducted using mass spectrometry. EV samples derived from both normoxia-and hypoxia-cultured MSCs were analyzed to identify differentially expressed proteins and their associated biological functions and localizations (FIG. 4A-4C).

The proteomic analysis revealed distinct differences in the protein cargo of hypoxia MSC-EVs relative to normoxia MSC-EVs. Multiple proteins were found to be significantly upregulated in the hypoxia EV group, many of which are associated with immunomodulation, antiviral response, cellular stress resistance, and tissue regeneration (FIG. 4A). These proteins were not observed at comparable levels in normoxia-derived EVs, indicating that hypoxic preconditioning selectively enriches for bioactive components relevant to lung injury repair.

Gene ontology (GO) analysis further revealed that the upregulated proteins in hypoxia MSC-EVs were predominantly localized to extracellular vesicles, cytoplasmic membranes, and exosomal compartments (FIG. 4B). From a functional perspective, these proteins were found to be involved in key biological processes including cytokine binding, receptor-mediated signaling, wound healing, oxidative stress regulation, and RNA binding (FIG. 4C).

The proteomic distinctions observed between hypoxia and normoxia MSC-EVs provide molecular-level validation for the enhanced functional performance of hypoxia EVs demonstrated in vitro and in vivo. Notably, the differential expression profile was not predictable based on prior studies of normoxia MSC-EVs and highlights the importance of hypoxic conditioning in modulating the therapeutic cargo of EVs. These findings support the inventive concept that simple preconditioning of MSCs under hypoxic conditions yields structurally and functionally distinct EVs with superior regenerative and antiviral potential.

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated.

What is claimed is:

1. An extracellular vesicle (EV) composition comprising a population of EVs isolated from one or more mesenchymal stromal cells (MSCs) cultured under chemically induced hypoxic conditions, wherein the one or more MSCs are exposed to a hypoxia-mimetic agent prior to EV collection, the EVs exhibit a particle size distribution within a range of approximately 30 nm to approximately 150 nm in diameter and are characterized by a lipid bilayer membrane encapsulating a cytoplasmic protein cargo, wherein the cytoplasmic protein cargo comprises at least CD9 and hypoxia-inducible factor 1-alpha (HIF-1α), and is substantially free of GM130.

2. The EV composition of claim 1, wherein the MSCs are umbilical cord-derived mesenchymal stromal cells.

3. The EV composition of claim 1, wherein the one or more MSCs are exposed to cobalt(II) chloride at a concentration of 50 to 200 μM for 48 to 96 hours, or alternatively to hypoxia-mimetics such as deferoxamine at a concentration of 50 to 100 μM or dimethyloxaloylglycine at a concentration of 0.5 to 1 mM.

4. The EV composition of claim 1, wherein the EVs are obtained by sequential ultracentrifugation and 0.22 μm membrane filtration from conditioned medium.

5. The EV composition of claim 1, wherein the EVs comprise at least one protein selected from the group consisting of Apolipoprotein E (APOE), Fibrinogen Gamma Chain (FGG), and Neuronal Pentraxin-1 (NPTX1), wherein the expression level of the at least one protein is at least 2-fold higher than that in EVs derived from normoxia-cultured MSCs.

6. The EV composition of claim 1, wherein the EVs, when applied to H5N1-infected alveolar epithelial cells, reduce mRNA expression levels of at least one cytokine selected from TNF-α, IFN-β, and RANTES by at least 2-fold relative to untreated infected cells.

7. The EV composition of claim 1, wherein the EVs upregulate expression of ion transporters selected from the group consisting of epithelial sodium channels (ENaC), CFTR, and Na⁺/K⁺ ATPase.

8. The EV composition of claim 1, wherein the EVs induce ISG15 mRNA expression by at least 2-fold in virus-infected alveolar epithelial cells.

9. The EV composition of claim 1, wherein the EVs are further formulated in a pharmaceutically acceptable carrier suitable for intravenous administration.

10. The EV composition of claim 1, wherein the EVs are formulated in a dosage form selected from the group consisting of:
a lyophilized powder comprising one or more cryoprotectants selected from the group consisting of trehalose, sucrose, and mannitol;
a nebulizable aqueous solution formulated for pulmonary administration; and
one or more polymeric microparticles comprising the EVs encapsulated in a biodegradable polymer selected from the group consisting of poly(lactic-co-glycolic acid) (PLGA), chitosan, and hyaluronic acid.

11. A method for treating acute lung injury in a subject in need thereof, comprising administering to the subject an effective amount of the extracellular vesicle (EV) composition of claim 1, wherein the lung injury is caused by viral infection, wherein the EVs are derived from mesenchymal stromal cells (MSCs) cultured under chemically induced hypoxia using a hypoxia-mimetic agent.

12. The method of claim 11, wherein the MSCs are exposed to cobalt(II) chloride at a concentration of 50 to 200 μM for 48 to 96 hours.

13. The method of claim 11, wherein the EV composition is administered via an intravenous, intranasal, or intratracheal route.

14. The method of claim 11, wherein the EVs reduce viral burden in a lung tissue by at least 50% relative to an untreated control.

15. The method of claim 11, wherein the EVs reduce expression of TNF-α and RANTES by at least 2-fold compared to an untreated control.

16. The method of claim 11, wherein the EVs restore alveolar fluid clearance (AFC) and reduce alveolar protein permeability (APP) within 24 hours post-administration.

17. The method of claim 11, wherein the EVs increase ISG15 expression by at least 2-fold in infected alveolar epithelial cells.

18. The method of claim 11, wherein the EV composition is co-administered with one or more antiviral agents comprising oseltamivir, zanamivir, or ribavirin.

19. The method of claim 11, wherein the effective amount of the EV composition comprises a dose of approximately 1×10⁸ to 1×10¹² EV particles per administration.

20. The method of claim 11, wherein the EV composition is administered in multiple doses over a period of 3 to 10 days following onset of viral infection symptoms.
